(19) United States
(12) Patent Application Publication
(10) Pub. No.: US 2005/0287118 A1
(43) Pub. Date: Dec. 29, 2005

(54) Bacterial plasmid with immunological adjuvant function and uses thereof

(75) Inventors: Maoxin Tian, Burlingame, CA (US); William J. Rutter, Burlingame, CA (US); Mark Selby, Burlingame, CA (US)

Correspondence Address: ROBINS & PASTERNAK, 1731 EMBARCADERO ROAD, SUITE 230, PALO ALTO, CA, 94303, US

(73) Assignee: Epitomics, Inc.

(21) Appl. No.: 10/997,747

(22) Filed: Nov. 23, 2004

Related U.S. Application Data
(60) Provisional application No. 60/525,311, filed on Nov. 26, 2003.
(60) Provisional application No. 60/528,468, filed on Dec. 9, 2003.

(51) Int. Cl. A61K 48/00; C07H 21/04; C12P 21/06; C12P 21/04; C07K 16/18; C12N 5/06

(52) U.S. Cl. 424/93.2; 435/69.1; 435/320.1; 435/326; 530/387.1; 536/23.53

(57) ABSTRACT

Plasmid adjuvant compositions and methods for enhancing an immune response to a coadministered immunogen are described. The plasmid adjuvants include a combination of cytokines and chemokines designed to elicit an enhanced immune response. Particular combinations can be provided to generate a Th1 and/or a Th2 immune response.

This application claims benefit under 35 U.S.C. § 119(e) of provisional application 60/528,468, filed on Dec. 9, 2003, entitled BACTERIAL PLASMID WITH IMMUNOLOGICAL ADJUVANT FUNCTION AND USES THEREOF, and provisional application 60/525,311, filed on Nov. 26, 2003, entitled A HIGH THROUGHPUT METHOD OF DNA IMMUNOGEN PREPARATION AND IMMUNIZATION, all of which applications are hereby incorporated by reference in their entireties.

### TECHNICAL FIELD

The invention relates generally to the field of immunization. In particular, the invention relates to plasmid adjuvant compositions and methods for enhancing an immune response to a coadministered immunogen.

### BACKGROUND

Numerous vaccine formulations which include attenuated pathogens or subunit protein antigens, have been developed. Additionally, antigen-encoding DNA vaccines have been produced that can be directly introduced into the body where the antigen is produced de novo, circumventing the need for protein antigen expression and purification.

Whether purified proteins or antigen-expressing DNA are used in immunization, immunological adjuvants are often coadministered to augment cell-mediated and humoral immune responses. Such adjuvants include depot adjuvants, compounds which adsorb and/or precipitate administered antigens and which serve to retain the antigen at the injection site. Typical depot adjuvants include aluminum compounds and water-in-oil emulsions. However, the above-described adjuvants, although increasing antigenicity, often provoke severe persistent local reactions, such as granulomas, abscesses and scarring, when injected subcutaneously or intramuscularly. Moreover, traditionally used adjuvants, such as Freund's adjuvant and metal or detergent-based adjuvants, are empirically formulated. Thus, the mechanisms and targets of these adjuvants are often unknown. See, e.g., Billiau et al., J. Leukoc. Biol. (2001) 70:849-860; Canki et al., AIDS Res. Hum. Retroviruses (1994) 10 Suppl. 2:S99-103. Other adjuvants, such as lipopolysacharrides and muramyl dipeptides, can elicit pyrogenic responses upon injection and/or Reiter's symptoms (influenza-like symptoms, generalized joint discomfort and sometimes anterior uveitis, arthritis and urethritis). Accordingly, there is a continued need for effective and safe adjuvants for use in a variety of pharmaceutical compositions and vaccines.

Various cytokines and chemokines (a class of cytokines with chemoattractant properties), ligands for toll-like receptors (TLRs) of the innate immune system, such as CpG DNA, poly(I:C), peptidylglycans, etc. have also been used as adjuvants. For example, type 1 interferon inducers, such as double-stranded RNA (dsRNA) have been reported to display adjuvant activity. See, e.g., Le Bon et al., Immunity (2001) 14:461-470. Bacterial DNA, but not vertebrate DNA, has direct immunostimulatory effects on peripheral blood mononuclear cells (PBMC) in vitro (Krieg et al., Proc. Natl. Acad. Sci. (1995) 374:546-549). This lymphocyte activation appears to be due to unmethylated CpG dinucleotides which are present at the expected frequency in bacterial DNA (1/16), but are under-represented and methylated in vertebrate DNA. For a description of the use of CpG oligonucleotides as immunostimulators see, e.g., Klinman, D. M., Expert Rev. Vaccines (2003) 2:305-315 and U.S. Pat. Nos. 6,406,705, 6,339,068, 6,207,646, 6,194,388, 6,218,371 and 6,429,199, which patents are incorporated herein by reference in their entireties.

Despite the continued discovery and understanding of the mechanisms of action of such adjuvants, conventional vaccine compositions often fail to provide adequate protection against the targeted pathogen. Thus, there is a continued need for the development of safe and effective adjuvants for use in immunogenic compositions for antibody production, DNA immunization, or for use in traditional vaccines for treating and/or preventing disease.

### SUMMARY OF THE INVENTION

The present invention addresses this need. In particular, the invention provides novel plasmid adjuvants and plasmid adjuvant systems that enhance immunological responses to coadministered antigens. Thus, the invention is useful for both monoclonal and polyclonal antibody production, as well as for vaccination against a wide variety of pathogens and diseases in animals, including humans. Moreover, the plasmid adjuvants can be designed to produce particular immune responses.

Accordingly, in one embodiment, the invention is directed to a plasmid adjuvant capable of enhancing the immunological response to a coadministered immunogen. In certain embodiments, the plasmid adjuvant comprises in 5′ to 3′ order, a promoter sequence; a sequence encoding a chemokine/cytokine fusion protein; an internal ribosome entry sequence (IRES); and a sequence encoding a CD40 ligand. The CD40 ligand can be a secreted or membrane-bound form.

In particular embodiments, the chemokine/cytokine fusion comprises a sequence encoding a secondary lymphoid tissue chemokine (SLC) fused to a sequence encoding an IL-4. The SLC sequence can be fused to the 5′ or 3′ terminus of the IL-4 sequence (i.e., the SLC sequence can either precede or follow the IL-4 sequence in the fusion). Other chemokine and/or cytokine sequences can be present in addition to, or in place of the SLC, IL-4 and/or the CD40 ligand. For example, a GM-CSF sequence can be present in addition to or in place of the SLC, IL-4 and/or the CD40 ligand sequence. Moreover, other chemokines that attract T- or B-lymphocytes or antigen presenting cells (APCs) can be present. The various cytokines/chemokines can be selected based on the species that will be immunized, as discussed more fully below.

In certain embodiments, the sequence encoding the chemokine/cytokine fusion is operatively linked to the promoter sequence such that protein expression occurs. In other embodiments, the above sequences, including a promoter sequence, are present but the plasmid is a non-coding plasmid, e.g., where the chemokine/cytokine coding sequence is in a reversed orientation.

In another embodiment, the plasmid adjuvant comprises the sequence depicted in FIGS. 4A-4B, or a contiguous sequence of nucleotides with at least 75% identity thereto, such as at least 80, or 85, or 90 or 95 or 98% sequence identity thereto.

In another embodiment, the invention is directed to a plasmid adjuvant system capable of enhancing the immunological response to a coadministered immunogen, wherein the plasmid adjuvant system comprises a polynucleotide encoding an SLC, a polynucleotide encoding an IL-4 and a polynucleotide encoding a CD40-ligand. The polynucleotides can be provided in cis, on the same plasmid or in trans, on separate plasmids. In this configuration, the SLC and IL-4 can either be fused, or provided separately.

In additional embodiments, the invention is directed to compositions comprising the plasmid adjuvants and adjuvant systems as described above, in combination with a pharmaceutically acceptable excipient. In certain embodiments, the compositions also include an immunogen, such as a protein or DNA immunogen.

In yet further embodiments, the invention is directed to a method of enhancing an immunological response in a vertebrate subject comprising administering an immunogen to a vertebrate subject, and coadministering a plasmid adjuvant or plasmid adjuvant system as described above to the vertebrate subject. The immunogen and plasmid adjuvant or adjuvant system can be present in the same composition or in different compositions. If present in different compositions, the adjuvant composition can be administered prior to, concurrent with, or subsequent to the immunogen. Moreover, the administering can be at the same or different sites.

In additional embodiments, the invention is directed to a method of inducing a Th2 immune response in a subject by administering to the subject a plasmid adjuvant or plasmid adjuvant system as described above.

These and other embodiments of the subject invention will readily occur to those of skill in the art in view of the disclosure herein.

### BRIEF DESCRIPTION OF THE FIGURES

FIGS. 1A-1D show a representative strategy for generating an exemplary plasmid adjuvant termed “SlcIl4IresCD40LpORF.” FIG. 1A shows the production of a BglII-IRES-NcoI fragment; FIG. 1B shows the production of an SLC-IL-4 fusion construct as an NcoI-BglII fragment; FIG. 1C shows the coding plasmid SlcIl4IresCD40LpORF where the SLC-IL-4 fusion is operatively linked to the EF1 alpha promoter such that the cytokines can be expressed; FIG. 1D shows a non-coding conformation of the plasmid where the NcoI-NcoI fragment of SLC-IL4-IRES is in the 3′ to 5′ direction with respect to the EF1 alpha promoter so that none of the cytokines are capable of expression.

FIG. 2 shows the results of a nucleic acid immunization experiment where animals were administered a DNA immunogen encoding PSA, and adjuvant plasmids of the invention. A=animals administered the immunogen plus the adjuvant plasmid shown in FIG. 1C; B=animals administered the immunogen plus the non-coding variant shown in FIG. 1D; C=animals given an empty plasmid lacking the promoter and the cytokine segment; D=animals immunized with the immunogen without any plasmid DNA; E=animals given a control immunization.

FIG. 3 depicts the results of a nucleic acid immunization experiment wherein animals were administered a DNA immunogen encoding MTF, and adjuvant constructs of the invention.

FIGS. 4A-4B depict the sequence of a plasmid SlcIl4IresCD40LpORF (SEQ ID NO:1). The SLC gene spans nucleotide positions 690 to 1088; the IL-4 gene spans nucleotide positions 1090 to 1452; the SLC-IL-4 fusion spans nucleotide positions 690 to 1452; the IRES spans nucleotide positions 1459 to 2007; the CD40 ligand sequence spans positions 2010-2792.

FIG. 5 shows the nucleotide and corresponding amino acid sequence of a representative SLC-IL-4 fusion (SEQ ID NOS:2 and 3).

FIGS. 6A-6B depict the sequence of the non-coding version of plasmid SlcIl4IresCD40LpORF (SEQ ID NO:4). The segment from nucleotide position 690 to nucleotide position 2007 of SEQ ID NO:1, which contains the SLC-IL-4 fusion and IRES sequences, is reversed in non-coding orientation.

### DETAILED DESCRIPTION OF THE INVENTION

The practice of the present invention will employ, unless otherwise indicated, conventional methods of molecular biology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

1. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a coding sequence” includes a mixture of two or more coding sequences, and the like.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

The terms “analog” and “mutein” refer to biologically active derivatives of the reference molecule, or fragments of such derivatives, that retain desired activity, such as immunoreactivity in the assays described herein. In general, the term “analog” refers to compounds having a native polypeptide sequence and structure with one or more amino acid additions, substitutions (generally conservative in nature, or in the case of a modified MEFA, generally non-conservative in nature at the NS3 proteolytic cleavage sites) and/or deletions, relative to the native molecule, so long as the modifications do not destroy immunogenic activity. The term “mutein” refers to polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same immunoactivity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “fragment” is intended a polypeptide consisting of only a part of the intact full-length polypeptide sequence and structure. The fragment can include a C-terminal deletion an N-terminal deletion, and/or an internal deletion of the native polypeptide.

An “antigen” refers to a molecule, such as a polypeptide as defined above, containing one or more epitopes (either linear, conformational or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A-T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. Similarly, an oligonucleotide or polynucleotide that expresses an antigen or antigenic determinant in vivo, such as in nucleic acid immunization applications, is also included in the definition of antigen herein. For purposes of the present invention, immunogens can be derived from any organism for which an immune response is desired, including immunogens derived from viruses, bacteria, fungi, parasites and the like, as described more fully below.

By “immunogenic fragment” is meant a fragment of the reference polypeptide that includes one or more epitopes and thus elicits one or more of the immunological responses described herein. An “immunogenic fragment” of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains the ability to elicit an immunological response as defined herein.

The term “epitope” as used herein refers to a sequence of at least about 3 to 5, preferably about 5 to 10 or 15, and not more than about 500 amino acids (or any integer therebetween), which define a sequence that by itself or as part of a larger sequence, elicits an immunological response in the subject to which it is administered. Often, an epitope will bind to an antibody generated in response to such sequence. There is no critical upper limit to the length of the epitope, which may comprise nearly the full-length of the protein sequence, or even a fusion protein comprising two or more epitopes from the molecule in question. An epitope for use in the subject invention is not limited to a polypeptide having the exact sequence of the portion of the parent protein from which it is derived. For example, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1985) Proc. Natl. Acad. Sci. USA 82:178-182; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular immunogen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Recent methods of measuring cell-mediated immune response include measurement of intracellular cytokines or cytokine secretion by T-cell populations, or by measurement of epitope specific T-cells (e.g., by the tetramer technique)(reviewed by McMichael, A. J., and O'Callaghan, C. A., J. Exp. Med. (1998) 187:1367-1371; Mcheyzer-Williams et al, Immunol. Rev. (1996) 150:5-21; Lalvani et al., J. Exp. Med. (1997) 186:859-865.

Thus, an immunological response as used herein may be one that stimulates the production of antibodies (e.g., neutralizing antibodies that block viruses from entering cells and/or replicating by binding to the pathogens, typically protecting cells from infection and destruction). The antigen of interest may also elicit production of CTLs. Hence, an immunological response may include one or more of the following effects: the production of antibodies by B-cells; and/or the activation of suppressor T-cells and/or 6y T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. (See, e.g., Montefiori et al., J. Clin Microbiol. (1988) 26:231-235; Dreyer et al., AIDS Res Hum Retroviruses (1999) 15:1563-1571).

An “immunogenic composition” is a composition that comprises an immunogenic molecule, such as a protein immunogen or nucleic acid encoding an immunogen, where administration of the composition to a subject results in the development in the subject of a humoral and/or a cellular immune response to the antigenic molecule of interest. The immunogenic composition can be introduced directly into a recipient subject, such as by injection, inhalation, oral, intranasal and mucosal (e.g., intrarectally or intravaginally) administration.

An “immunological adjuvant” refers to a plasmid adjuvant or plasmid adjuvant system that potentiates an immunological response in the subject to which it is administered. The adjuvant can be incorporated into or administered with the immunogen or administered in a separate composition. Alternatively, the adjuvant can be administered without an accompanying antigen to stimulate nonspecific immunity.

An adjuvant composition comprising an plasmid adjuvant or plasmid system as described herein “enhances” or “increases” the immune response, or displays “enhanced” or “increased” immunogenicity vis-a-vis a selected immunogen when it possesses a greater capacity to elicit an immune response than the immune response elicited by an equivalent amount of the immunogen when delivered without the plasmid adjuvant. Such enhanced immunogenicity can be determined by administering the immunogen and adjuvant, and controls to animals and comparing antibody titers (if the immunogen stimulates an antibody response) against the two using standard assays such as radioimmunoassay and ELISAs, well known in the art. Methods for determining the presence of a cell-mediated immune response are described above. The adjuvant may enhance the immunological response by making the immunogen more strongly immunogenic or by lowering the dose of immunogen necessary to achieve an immune response in the subject to which it is administered.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “coding sequence” or a sequence which “encodes” a selected polypeptide, is a nucleic acid molecule which can be transcribed and translated into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. As used herein, the terms “expression cassette” or “expression construct” do not necessarily imply that the cassette or construct is present in a plasmid.

By “nucleic acid immunization” is meant the introduction of a nucleic acid molecule encoding one or more selected immunogens into a host cell, for the in vivo expression of the immunogen. The nucleic acid molecule can be introduced directly into a recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the immunogen encoded by the nucleic acid molecule.

The terms “effective amount” or “pharmaceutically effective amount” of an immunogenic composition and/or adjuvant composition, as provided herein, refer to a nontoxic but sufficient amount of the composition to provide the desired response, such as an immunological response, and optionally, a corresponding therapeutic effect. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

The term “treatment” as used herein refers to either (1) the prevention of infection or reinfection (prophylaxis), or (2) the reduction or elimination of symptoms of the disease of interest (therapy).

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

2. Modes of Carrying out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that certain plasmid adjuvants and plasmid adjuvant systems comprising chemokine and other cytokine sequences, display strong immunity-enhancing properties. The sequences present can be picked in order to effect a desired immune response. In particular, cytokines play a role in directing the T cell response. Helper CD4+ T cells orchestrate the immune response of mammals through production of soluble factors that act on other immune system cells, including B and other T cells. Most mature CD4+ T helper cells express one of two cytokine profiles: Th1 or Th2. Th1 cells secrete IL-2, IL-3, IFN-gamma, GM-CSF and high levels of TNF-alpha. Th2 cells express IL-3, IL-4, IL-5, IL-6, IL-9, IL-10, IL-13, GM-CSF and low levels of TNF-alpha. The Th1 subset promotes both cell-mediated immunity, and humoral immunity that is characterized by immunoglobulin class switching to IgG2a in mice. Th1 responses may also be associated with delayed-type hypersensitivity and autoimmune disease. The Th2 subset induces primarily humoral immunity and induces class switching to IgG1 and IgE.

Cytokines have been shown to influence commitment to Th1 or Th2 profiles. IL-12 and IFN-gamma are positive Th1 and negative Th2 regulators. IL-12 promotes IFN-gamma production, and IFN-gamma provides positive feedback for IL-12. IL-4 and IL-10 are involved in the establishment of the Th2 cytokine profile and down-regulate Th1 cytokine production. IL-13 has been shown to inhibit expression of inflammatory cytokines, including IL-12 and TNF-alpha by LPS-induced monocytes, in a way similar to IL-4. The IL-12 p40 homodimer binds to the IL-12 receptor and may antagonize IL-12 biological activity and thus blocks the pro-Th1 effects of IL-12 in some animals.

Thus, by manipulating the cytokines present in the plasmid adjuvants of the invention, a primarily Th1 and/or Th2 immune response can be generated.

The adjuvant compositions can be introduced into the subject using any of various DNA delivery techniques, described more fully below. The adjuvant compositions can be used with immunogens, including polypeptide or DNA immunogens, as well as with inactivated or attenuated pathogens, to produce an immune response in the subject to which the compositions are delivered. The immune response can serve to protect against future infection, or can be for the production of antibodies, both polyclonal and monoclonal, for use as diagnostics, immunopurification reagents and the like.

In order to further an understanding of the invention, a more detailed discussion is provided below regarding the plasmid adjuvants, immunogens, as well as various nucleic acid delivery methods for use with the present invention.

The Plasmid Adjuvants

As explained above, the plasmid adjuvants and plasmid adjuvant systems of the invention include various cytokines and/or chemokines, that serve to enhance the immune response to a coadministered immunogen. In one embodiment of the invention, the various chemokines and cytokines are provided by a single plasmid, under the control of a single promoter. In other embodiments, the various substituents are provided as individual constructs, either on the same plasmid or on individual plasmids.

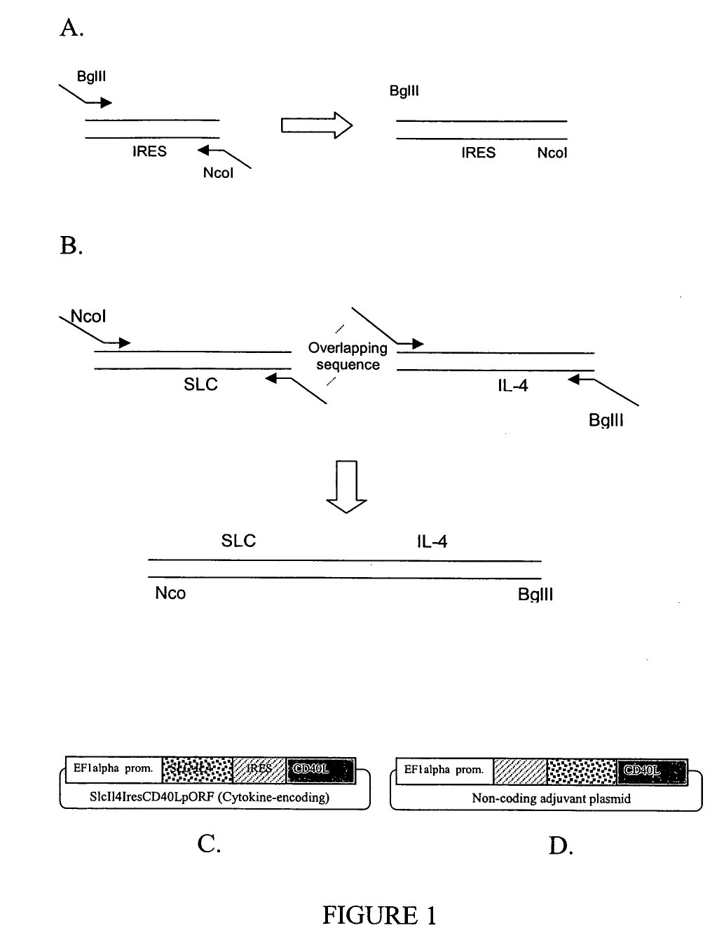

Representative plasmids for use with the present invention are depicted in FIGS. 1C and 1D. As shown in FIG. 1C, the plasmid includes a sequence encoding an SLC-IL-4 fusion operatively linked to the EF1alpha promoter, such that the SLC-IL-4 fusion is capable of expression. Also present is an IRES sequence and a coding sequence for a CD40 ligand. The presence of the CD40 ligand is especially desirable as the ligand is required for productive interactions between B-cells and helper T-cells. Moreover, the inventors have surprisingly found that the SLC-IL-4 coding sequence need not be expressed in order to impart enhanced immunogenicity to a coadministered immunogen. Thus, the fusion can be present in a reversed orientation, as discussed in the examples and shown in FIG. 1D, and still be capable of enhancing an immune response directed against a coadministered immunogen.

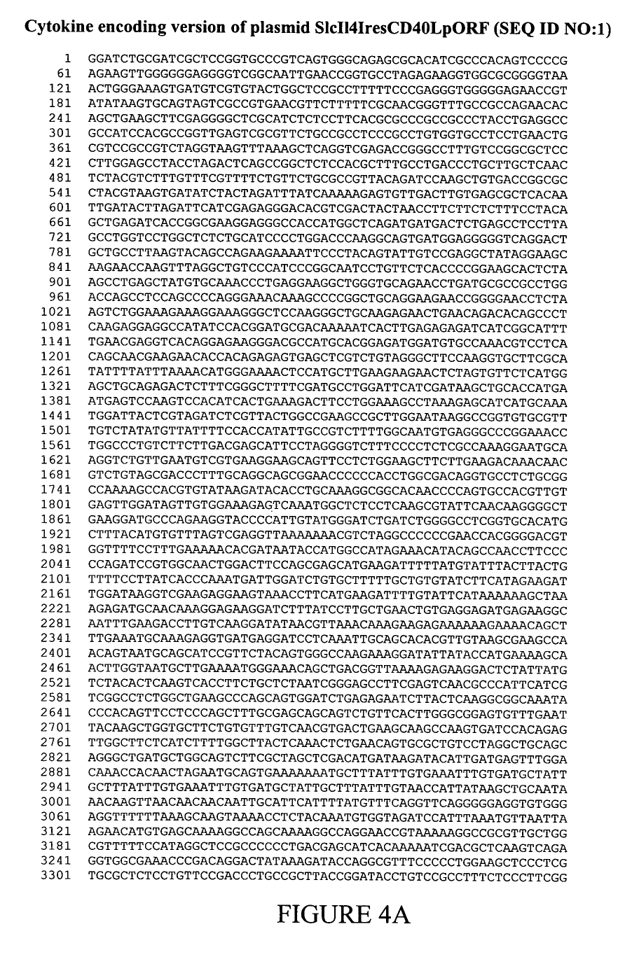

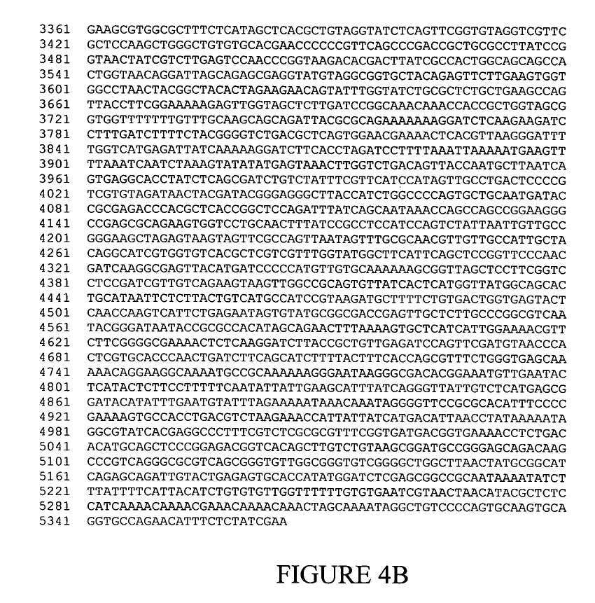

A representative plasmid adjuvant sequence is shown in FIGS. 4A-4B. However, the invention is not intended to be limited to this sequence. In particular, variants of this sequence will also find use herein, such as a plasmid adjuvant comprising a contiguous sequence of nucleotides with at least 75% identity thereto, such as at least 80, or 85, or 90 or 95 or 98% sequence identity thereto.

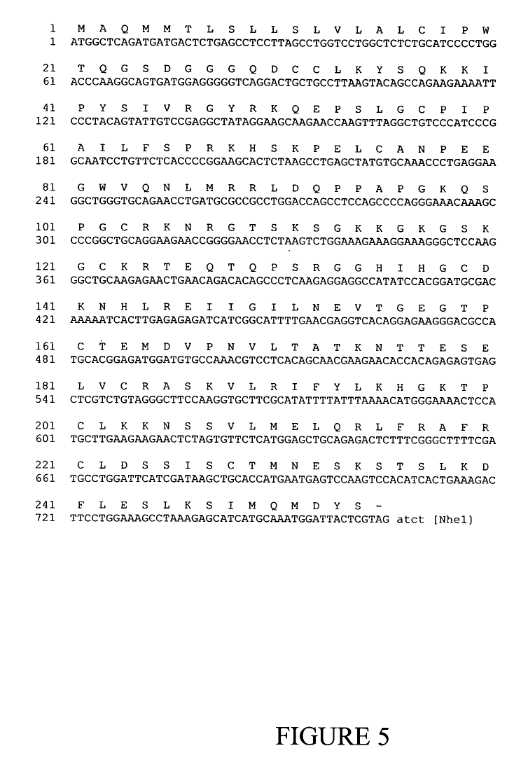

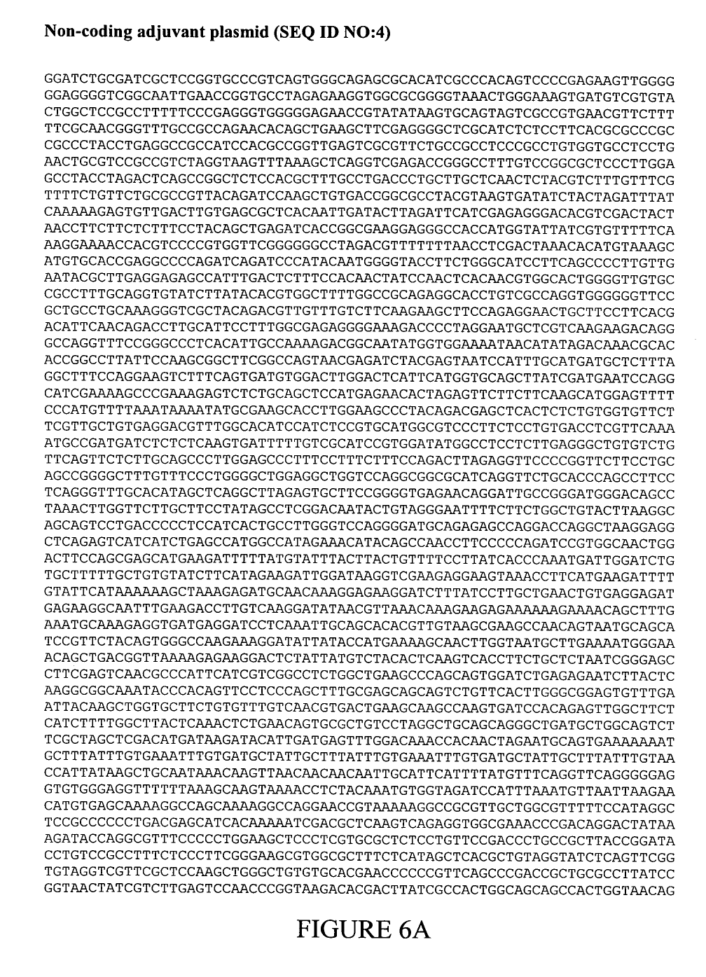

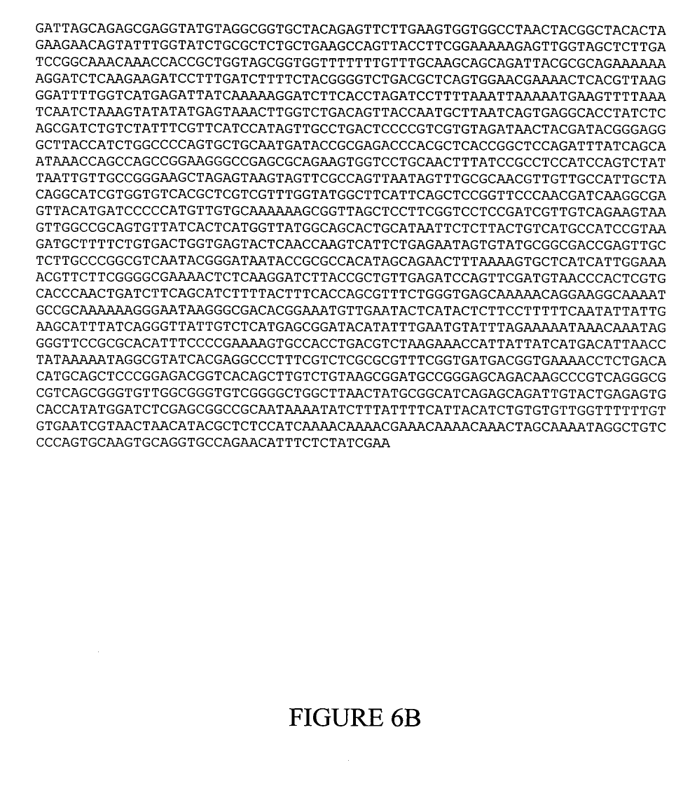

The selection of cytokines and chemokines for use with the subject compositions and methods is largely a matter of choice, dictated by the type of immune response desired. Thus, if a vigorous antibody response is desired, e.g., in order to generate a large number of antibodies, either for therapeutic use or for use as diagnostic or immunopurification agents, cytokines will be selected that provide a Th2-type response. Such cytokines include, without limitation, IL-4, IL-10, IL-5, IL-3, GM-CSF, IL-6, IL-9, IL-13, IL21, IL25 and the like, with IL-4, IL-10 and GM-CSF preferred. For a description of Th2-type cytokines see, e.g., Jarnicki et al., Curr. Opin. Pharmacol. (2003) 3:449-55. Moreover, it is desirable to include a chemokine in the constructs, such as SLC, and/or any other chemokine that attracts T- or B-lymphocytes or antigen presenting cells (APCs). In a preferred embodiment, the chemokine and cytokine are provided as a fusion, such as an SLC-IL-4 fusion. In this embodiment, the SLC (or other chemokine) sequence can be fused to the 5′ or 3′ terminus of the IL-4 (or other cytokine) sequence (i.e., the SLC sequence can either precede or follow the IL-4 sequence in the fusion). A representative SLC-IL-4 fusion is depicted in FIG. 5. However, variants of this sequence will also find use herein, such as plasmid adjuvants comprising SLC and IL-4 sequences from other species, or variants of this fusion that have a contiguous sequence of nucleotides with at least 75% identity to the sequence depicted in FIG. 5, such as at least 80, or 85, or 90 or 95 or 98% sequence identity thereto.

The above constituents can be from any source. In some cases it may be desirable to use sequences that encode cytokines and chemokines from the species to which the plasmid adjuvants will be administered. Moreover, the native molecules, as well as fragments and analogs thereof, which act in concert to enhance a desired immune response, are intended for use with the present invention.

Sequences for various cytokines and chemokines from a variety of species are well known in the art. Non-limiting examples of IL-4 sequences for use with the present invention include the IL-4 sequence shown in FIGS. 4 and 5, as well as the sequences described in NCBI accession numbers NM172348, AF395008, AB015021, X16710, A00076, M23442, M13982, NM000589 (all human sequences); BC027514, NM021283, AF352783, M25892 (mouse sequences); NM173921, AH003241, M84745, M77120, U14160 (bovine sequences); AY130260, AF097321, L26027, AY339648, AY083268, AY339647, AY339646, AY339645, AY339644, U19838 (nonhuman primate sequences); AY096800, AF172168, Z11897, M96845 (ovine sequences); AF035404, AF305617 (equine sequences); AF083270, AF239917, AF187322, AF054833, AF104245 (canine sequences); X16058 (rat); AF046213 (hamster); L07081 (cervine); U39634, X87408 (feline); X68330, L12991 (porcine sequences); U34273 (goat); AB020732 (dolphin); L37779 (gerbil); AF068058, AF169169 (rabbit sequences); AB107648 (llama and camel); AF542141 (viral sequence).

Representative, non-limiting examples of IL-10 sequences for use with the present invention include the sequences described in NCBI accession numbers NM000572, U63015, AF418271, AF247603, AF247604, AF247606, AF247605, AY029171, UL16720 (all human sequences); NM012854, L02926, X60675 (rat); NM010548, AF307012, M37897, M84340 (all mouse sequences); U38200 (equine); U39569, AF060520 (feline sequences); U00799 (bovine); U11421, Z29362 (ovine sequences); L26031, L26029, AF294758 (nonhuman primate sequences); U33843 (canine); AF088887, AF068058 (rabbit sequences); AF012909, AF120030 (woodchuck sequences); AF026277 (possum); AF097510 (guinea pig); U11767 (deer); L37781 (gerbil); AB107649 (llama and camel).

Representative, non-limiting examples of SLC sequences for use with the present invention include the SLC sequences shown in FIGS. 4 and 5, as well as sequences described in NCBI accession numbers AY358887, NM002989, AB002409, BC027918 (all human sequences); NM011124, NM023052, BC025974, BC038120, BC028747, NM011335 (all mouse sequences).

Representative, non-limiting examples of CD40 ligand sequences for use with the present invention include the CD40 ligand sequences shown in FIG. 4, as well as sequences described in NCBI accession numbers L07414, D31797, E09514, E09513, E09512, E09511, E09510, X67878, X96710 (all human sequences); AF344853, AF344841, AF344844, AF344859, AF344860 (nonhuman primate sequences); X65453 (mouse); NM053353, AF013985 (rat); AF079105 (cat); AY333790, AF086711 (dog); AT243435 (chicken); Z48469 (bovine); AF263915 (porcine).

Representative, non-limiting examples of GM-CSF sequences for use with the present invention sequences described in NCBI accession numbers E02975, E02287, E01817, E00951, M11220, A11763, X03021 (all human sequences); AY234216, AY007376 (nonhuman primate sequences); AF38736 (gerbil); AF053007 (cat); S49738 (dog); X03020, X02333, X03019, X03221, NM009969, E09685, E09574, E00950 (mouse); X55991, X53561 (ovine); NM174027, U22385 (bovine); AY116504, D21074, U67318, U67175 (porcine).

As explained above, an IRES sequence is preferably present when the various constituents of the plasmid adjuvant system are provided on the same plasmid. This allows expression of the various cytokines under the control of a single promoter. Such constructs are referred to in the art as “dicistronic” or “bicistronic.” The IRES element permits the translation of two or more open reading frames from a single messenger RNA, one encoding the chemokine/cytokine fusion protein of interest and the other encoding another cytokine, such as the CD40 ligand. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22 150-161.

A multitude of IRES sequences are known and include sequences derived from a wide variety of viruses, such as from leader sequences of picomaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. Nov. 26, 2003), an IRES element from the foot and mouth disease virus (Ramesh et al., Nucl. Acid Res. (1996) 24:2697-2700), a giardiavirus IRES (Garlapati et al., J. Biol. Chem. Nov. 12, 2003), and the like. A variety of nonviral IRES sequences will also find use herein, including but not limited to yeast IRESs, as well as the human angiotensin II type 1 receptor IRES (Martin et al., Mol. Cell Endocrinol. (2003) 212:51-61). These elements are readily commercially available in plasmids sold by e.g., Invivogen (San Diego, Calif.) and Stratagene (La Jolla, Calif.).

Polynucleotides encoding the desired cytokines, chemokines and IRESs for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. The gene of interest can also be produced synthetically, rather than cloned, based on the known sequences. The molecules can be designed with appropriate codons for the particular sequence. The complete sequence is then assembled from overlapping oligonucleotides prepared by standard methods and assembled into a complete coding sequence. See, e.g., Edge, Nature (1981) 292:756; Nambair et al., Science (1984) 223:1299; and Jay et al., J. Biol. Chem. (1984) 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. One method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239:1534-1536), and enzymatic filling-in of gapped oligonucleotides using T₄ DNA polymerase (Queen et al., Proc. Natl. Acad. Sci. USA (1989) 86:10029-10033) can be used to provide molecules for use in the subject methods.

If the sequences are to be provided together in a plasmid, they can be designed to contain restriction sites compatible with a host vector or plasmid and the various polynucleotides can be ligated into a plasmid vector which contains appropriate control and regulatory sequences such that the coding sequences can be transcribed in vivo to produce the cytokines, if desired. As explained above and shown in the examples, however, expression of the cytokines is not necessary in order to achieve an enhanced immune response.

If present, the promoter for use in the plasmid adjuvant is one capable of directing transcription of the various cytokines in a vertebrate subject when the chemokine/cytokine fusion is operably linked thereto. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other promoters, such as but not limited to the EF1alpha promoter, or a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Transcription terminator/polyadenylation signals may also be present in the constructs. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same.

The various nucleotide sequences and the desired plasmid which has been digested with the particular restriction enzyme of interest are then ligated together using a DNA ligase and techniques well known in the art to produce an expression vector, with the coding sequence and control sequences positioned and oriented such that the coding sequence is transcribed under the “control” of the control sequences (i.e., RNA polymerase which binds to the DNA molecule at the control sequences transcribes the coding sequence).

Immunogens

As explained above, immunogens for use with the adjuvant compositions can include immunogens of viral, bacterial, mycobacterial, fungal, parasitic, etc. origin. Non-limiting examples of viral pathogens that affect humans and/or nonhuman vertebrates from which immunogens can be derived, or which can be provided in attenuated or inactivated form include retroviruses, RNA viruses and DNA viruses. The group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses from which immunogens can be derived include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including the SARS virus, Infectious Bronchitis Virus (IBV), Mouse Hepatitis virus, Human enteric corona virus, and Feline infectious peritonitis (Feline coronavirus).

Illustrative DNA viruses from which immunogens can be derived include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex virus Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Non-limiting examples of bacterial pathogens from which immunogens can be derived include both gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pylori, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of infectious fungi from which immunogens can be derived include: Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e. protists) include Toxoplasma gondii.

Other medically relevant microorganisms have been described extensively in the literature. See, e.g. C. G. A Thomas, Medical Microbiology, Bailliere Tindall, Great Britain 1983, the entire contents of which is hereby incorporated by reference.

Although many of the pathogens described above relate to human disorders, the invention is also useful for treating other nonhuman vertebrates. Nonhuman vertebrates are also capable of developing infections which can be prevented or treated using the plasmid adjuvants disclosed herein. For instance, in addition to the treatment of infectious human diseases, the methods of the invention are useful for treating infections of animals.

For example, birds, cattle, horses and other farm animals are susceptible to infection. Diseases which affect these animals can produce severe economic losses. Thus, the compositions and methods of the invention can be used to protect against infection in livestock, such as cows, horses, pigs, sheep, and goats. For example, the compositions and methods can be used to protect against shipping fever, bovine viral diarrhea virus (BVDV), hog cholera virus (HOCV), sheep border disease virus (BDV), Equine herpesviruses (EHV), and visna-maedi. Cats, both domestic and wild, are also susceptible to infection with a variety of microorganisms. Thus, the invention is also useful for protecting pets against, for example, feline infectious peritonitis (FIP), feline leukemia virus (FeLV), feline sarcoma virus (FeSV), endogenous type C oncomavirus (RD-114), feline syncytia-forming virus (FeSFV), and feline T-lymphotropic lentivirus (also referred to as feline immunodeficiency).

Viral, bacterial and parasitic diseases in fin-fish, shellfish or other aquatic life forms pose a serious problem for the aquaculture industry. The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals. Thus, the present compositions can also be used to vaccinate fish. Nucleic acid-based vaccinations for fish are described in, e.g., U.S. Pat. No. 5,780,448, incorporated herein by reference in its entirety. Aquaculture species include but are not limited to fin-fish, shellfish, and other aquatic animals.

It is readily apparent that the subject plasmid adjuvants will find use for the delivery of a wide variety of immunogens to both human and nonhuman organisms. These immunogens can be provided as attenuated, inactivated or subunit vaccine compositions. Additionally, the immunogens can be provided in nucleic acid constructs for DNA immunization. Techniques for preparing DNA immunogens are well known in the art and described in, e.g., U.S. Pat. Nos. 5,399,346, 5,580,859, 5,589,466, incorporated by reference herein in their entireties.

Compositions and Administration

The invention provides compositions including the above-described plasmid adjuvants, as well as compositions including the immunogen. As explained above, the adjuvant can be provided as a plasmid adjuvant including the various components, or the components can be provided as individual constructs on the same vector or on separate vectors. The plasmid adjuvant (or components) and immunogen can be present in either the same or a different composition. If administered separately, the adjuvant can be given concurrently, prior to, or subsequent to immunization with the immunogen. If administered prior to immunization with the immunogen, the adjuvant formulations can be administered as early as 5-10 days prior to immunization, preferably 3-5 days prior to immunization and most preferably 1-3 or 2 days prior to immunization with the immunogen of interest. If administered separately, the adjuvant formulation can be delivered either to the same site of delivery as the immunogen composition or to a different delivery site.

If simultaneous delivery is desired, the immunogen can be included with the adjuvant. Generally, the immunogen and adjuvant can be combined by simple mixing, stirring, or shaking. Other techniques, such as passing a mixture of the two components rapidly through a small opening (such as a hypodermic needle) can also be used to provide the vaccine compositions.

The adjuvant and immunogen compositions can be formulated as injectables, either as liquid solutions or suspensions; solid forms suitable for solution in, or suspension in, liquid vehicles prior to injection may also be prepared. The compositions generally include excipients, such as water, saline, glycerol, dextrose, ethanol, or the like, singly or in combination, as well as substances such as wetting agents, emulsifying agents, or pH buffering agents.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art.

If desired, additional costimulatory molecules which improve immunogen presentation to lymphocytes, such as B7-1 or B7-2, or cytokines such as GM-CSF, IL-2, and IL-12, can be included in a composition of the invention.

Optionally, additional adjuvants can also be present, either in the same or different compositions. Additional adjuvants which can be used include, but are not limited to: (1) aluminum salts (alum), such as aluminum hydroxide, aluminum phosphate, aluminum sulfate, etc.; (2) oil-in-water emulsion formulations; (3) saponin adjuvants; (4) Complete Freund's Adjuvant (CFA) and Incomplete Freund's Adjuvant (IFA); (5) cytokines, such as interleukins (IL-1, IL-2, IL-4, IL-5, IL-6, IL-7, IL-12, interferons, macrophage colony stimulating factor (M-CSF), tumor necrosis factor (TNF), etc.; (6) detoxified mutants of a bacterial ADP-ribosylating toxin such as a cholera toxin (CT), pertussis toxin (PT), or an E. coli heat-labile toxin (LT); (7) oligonucleotides comprising CpG motifs (See, e.g., U.S. Pat. No. 6,207,646; Krieg et al. Nature (1995) 374:546 and Davis et al. J. Immunol. (1998) 160:870-876); as well as other immunostimulatory molecules.

Additionally, if the immunogen is provided as a nucleic acid, the immunogen and/or the plasmid adjuvant can be packaged in liposomes prior to delivery to the cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Immunogen compositions for use in the invention will comprise a therapeutically effective amount of the desired immunogen and any other of the above-mentioned components, as needed. By “therapeutically effective amount” in the context of the immunogen compositions is meant an amount of immunogen which will induce an immunological response, either for antibody production or for treatment or prevention of a particular disease or infection. Such a response will generally result in the development in the subject of an antibody-mediated and/or a secretory or cellular immune response to the composition. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell and/or γδT cell populations.

Plasmid adjuvant compositions will include an amount of plasmid effective to enhance the immune response to the coadministered immunogen. Such an amount can be readily determined by one of skill in the art and will depend on the animal being treated, the nature of the immune response desired, and the like.

Once formulated, the immunogen-containing compositions can be conventionally administered parenterally, e.g., by injection, either subcutaneously, intraperitoneally, intramuscularly or intravenously. Additional formulations suitable for other modes of administration include oral and pulmonary formulations, suppositories, and transdermal formulations, aerosol, intranasal, and sustained release formulations.

Dosage treatment may be a single dose schedule or a multiple dose schedule. The exact amount necessary will vary depending on the desired response, i.e., antibody production and/or a protective immune response; the subject being treated; the age and general condition of the individual to be treated; the capacity of the individual's immune system to synthesize antibodies; the degree of protection desired; the severity of the condition being treated; the particular macromolecule selected and its mode of administration, among other factors. An appropriate effective amount can be readily determined by one of skill in the art. A “therapeutically effective amount” will fall in a relatively broad range that can be determined through routine trials using in vitro and in vivo models known in the art.

The plasmid adjuvants and immunogenic compositions (if DNA immunogens are used) can be delivered using standard gene delivery protocols. Methods for gene delivery are known in the art. See, e.g., U.S. Pat. Nos. 5,399,346, 5,580,859, 5,589,466, incorporated by reference herein in their entireties. Nucleic acid can be delivered either directly to the subject or, alternatively, delivered ex vivo, to cells derived from the subject and the cells reimplanted in the subject.

A wide variety of methods can be used to deliver the constructs to cells. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, liposomes, peptoid delivery, or microinjection. See, e.g., Sambrook et al., supra, for a discussion of techniques for transforming cells of interest; and Felgner, P. L., Advanced Drug Delivery Reviews (1990) 5:163-187, for a review of delivery systems useful for gene transfer. Methods of delivering DNA using electroporation are described in, e.g., Selby et al., J. Immunol. (2000) 164:4635-4640; U.S. Pat. Nos. 6,132,419; 6,451,002, 6,418,341, 6233,483, U.S. Patent Publication No. 2002/0146831; and International Publication No. WO/0045823, all of which are incorporated herein by reference in their entireties.

Additionally, biolistic delivery systems employing particulate carriers such as gold and tungsten, are useful for delivering the constructs of the present invention. The particles are coated with the construct to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744.

The amount of DNA delivered will generally be about 1 μg to 500 mg of DNA, such as 5 μg to 100 mg of DNA, e.g., 10 μg to 50 mg, or 100 μg to 5 mg, such as 20 . . . 30 . . . 40 . . . 50 . . . 60 . . . 100 . . . 200 μg and so on, to 500 μg DNA, and any integer between the stated ranges.

Administration of the compositions can elicit an antibody titer and/or a cellular immune response in the animal that lasts for at least 1 week, 2 weeks, 1 month, 2 months, 3 months, 4 months, 6 months, 1 year, or longer. The compositions can also be administered to provide a memory response. If such a response is achieved, antibody titers may decline over time, however exposure to the particular immunogen results in the rapid induction of antibodies, e.g., within only a few days. Optionally, antibody titers can be maintained in a subject by providing one or more booster injections of the compositions, at e.g., 2 weeks, 1 month, 2 months, 3 months, 4 months, 5 months, 6 months, 1 year, or more after the primary injection.

Preferably, an antibody titer of at least 10, 100, 150, 175, 200, 300, 400, 500, 750, 1,000, 1,500, 2,000, 3,000, 5,000, 10,000, 20,000, 30,000, 40,000, 50,000 (geometric mean titer), or higher, is elicited, or any number between the stated titers, as determined using a standard immunoassay.

Antibodies

The plasmid adjuvants can be used with immunogens to produce immunogen-specific polyclonal and monoclonal antibodies. Such specific polyclonal and monoclonal antibodies specifically bind to the immunogen in question. Polyclonal antibodies can be produced by administering the plasmid adjuvants and immunogens to a mammal, such as a mouse, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies directed against specific epitopes encoded by the DNA immunogen can also be readily produced. Normal B cells from a mammal, such as a mouse, immunized with a DNA immunogen, can be fused with, for example, HAT-sensitive mouse myeloma cells to produce hybridomas. Hybridomas producing specific antibodies can be identified using RIA or ELISA and isolated by cloning in semi-solid agar or by limiting dilution. Clones producing the specific antibodies in question are isolated by another round of screening.

It may be desirable to provide chimeric antibodies, especially if the antibodies are to be used in preventive or therapeutic pharmaceutical preparations, such as for providing passive protection against infection, as well as in diagnostic preparations. Chimeric antibodies composed of human and non-human amino acid sequences may be formed from the mouse monoclonal antibody molecules to reduce their immunogenicity in humans (Winter et al. (1991) Nature 349:293; Lobuglio et al. (1989) Proc. Nat. Acad. Sci. USA 86:4220; Shaw et al. (1987) J Immunol. 138:4534; and Brown et al. (1987) Cancer Res. 47:3577; Riechmann et al. (1988) Nature 332:323; Verhoeyen et al. (1988) Science 239:1534; and Jones et al. (1986) Nature 321:522; EP Publication No. 519,596, published 23 Dec. 1992; and U.K. Patent Publication No. GB 2,276,169, published 21 Sep. 1994).

Antibody molecule fragments, e.g., F(ab′)₂, Fv, and sFv molecules, that are capable of exhibiting immunological binding properties of the parent monoclonal antibody molecule can be produced using known techniques. Inbar et al. (1972) Proc. Nat. Acad. Sci. USA 69:2659; Hochman et al. (1976) Biochem 15:2706; Ehrlich et al. (1980) Biochem 19:4091; Huston et al. (1988) Proc. Nat. Acad. Sci. USA 85(16):5879; and U.S. Pat. Nos. 5,091,513 and 5,132,405, to Huston et al.; and 4,946,778, to Ladner et al.

In the alternative, a phage-display system can be used to expand monoclonal antibody molecule populations in vitro. Saiki, et al. (1986) Nature 324:163; Scharf et al. (1986) Science 233:1076; U.S. Pat. Nos. 4,683,195 and 4,683,202; Yang et al. (1995) J Mol Biol 254:392; Barbas, III et al. (1995) Methods: Comp. Meth Enzymol 8:94; Barbas, III et al. (1991) Proc Natl Acad Sci USA 88:7978.

Once generated, the phage display library can be used to improve the immunological binding affinity of the Fab molecules using known techniques. See, e.g., Figini et al. (1994) J. Mol. Biol. 239:68. The coding sequences for the heavy and light chain portions of the Fab molecules selected from the phage display library can be isolated or synthesized, and cloned into any suitable vector or replicon for expression. Any suitable expression system can be used, including any of the various expression systems known in the art.

Antibodies which are directed against epitopes from a particular pathogen, are particularly useful for detecting the presence of that pathogen in a sample, such as a serum sample from an individual suspected of infection. An immunoassay may utilize one antibody or several antibodies. An immunoassay may use, for example, a monoclonal antibody directed towards a particular epitope, a combination of monoclonal antibodies directed towards multiple epitopes of a single pathogen, monoclonal antibodies directed towards epitopes of different pathogens, polyclonal antibodies directed towards the same pathogen, polyclonal antibodies directed towards different pathogens, or a combination of monoclonal and polyclonal antibodies. Immunoassay protocols may be based, for example, upon competition, direct reaction, or sandwich type assays using, for example, labeled antibody. The labels may be, for example, fluorescent, chemiluminescent, or radioactive.

The antibodies generated may also be used to isolate pathogens or antigens by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind pathogens or antigens from a biological sample, such as blood or plasma. The bound substances are recovered from the column matrix by, for example, a change in pH.

3. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

Enzymes were purchased from commercial sources, and used according to the manufacturers' directions.

In the isolation of DNA fragments, except where noted, and all DNA manipulations were done according to standard procedures. See, e.g., Sambrook et al., supra. Restriction enzymes, T₄ DNA ligase, DNA polymerase 1I, Klenow fragment, and other biological reagents can be purchased from commercial suppliers and used according to the manufacturers' directions. Sources for chemical reagents generally include Sigma Chemical Company, St. Louis, Mo.; Alrich, Milwaukee, Wis.; Roche Molecular Biochemicals, Indianapolis, Ind.

### EXAMPLE 1

### Production of Plasmid Adjuvant

A plasmid, named SlcIl4resCD40LpORF and a non-coding variant were constructed as follows. Plasmid SlcIl4resCD40LpORF has the configuration of EF1 alpha-SLC/IL4 fusion-IRES-CD40 ligand as depicted in FIG. 1C. The plasmid was produced by PCR linking the different fragments into pORF-mCD40L v.15 (InvivoGen, San Diego, Calif.). This plasmid contains the CD40 ligand sequence. The IL4 sequence was from pORF-mIL04 v.11 (InvivoGen). The SLC and IRES sequences were from pGT60mExodus2 v.02 (InvivoGen).

1a: Converting IRES into a BglII-NcoI Fragment:

The internal ribosome entry sequence (IRES) was amplified from an IRES-containing plasmid (pGT60mExodus2, Invivogen) using primers containing BglII and NcoI sites, as shown in FIG. 1A.

1b: The SLC-IL-4 Fusion Construct as an NcoI-BglII Fragment:

Mouse forms of SLC and IL-4 were amplified by PCR from, respectively, a SLC plasmid (pGT60mExodus2) from Invivogen and a IL-4 plasmid from ATCC (ATCC Accession No. 5645726). The primers were designed to contain the indicated restriction sites (NcoI, BglII) or an overlapping sequence which served to link the SLC and IL-4 sequences together. The resulting SLC and IL-4 fragments were subjected to a second round of PCR (an overlapping PCR), through the overlapping sequence, to produce a fusion open reading frame sequence (ORF). The ORF was flanked by two restriction sites, NcoI and BglII, that were engineered initially into the PCR primers (See, FIG. 1B). The nucleotide and corresponding amino acid sequence of the SLC-IL-4 fusion is shown in FIG. 5.

1c: Assembly of DNA Fragments to Form the Adjuvant Plasmid SlcIl4IrespORF:

Plasmid (pORF-mCD40L) containing the murine CD40 ligand sequence under the transcriptional control of EF-1alpha promoter was obtained from Invivogen. The NcoI-BglII segment of SLC-IL4 and the BglII-NcoI segment of IRES were cloned into the NcoI site of pORF-mCD40L. One of the two possible orientations allowed expression of SLC-IL4 fusion and CD40 ligand and was configured as EF1alpha-SLC-IL4-IRES-CD40L in the pORF vector, giving rise to the cytokine-encoding SlcIl4IresCD40LpORF (5.4 kb) (FIG. 1C). The other orientation, in which the NcoI-NcoI fragment of SLC-IL4-IRES was in the 3′ to 5′ direction with respect to the EF1 alpha promoter, did not permit expression of any of the cytokines and yet retained all the same nucleotides, giving rise to the non-coding version of SlcIl4IresCD40LpORF (5.4 kb) (FIG. 1D). Both versions of the SlcIl4IrespORF were verified by sequencing (the vector pORF, 3.2 kb in length, lacks any of the SLC-IL4, IRES or CD40L sequences). The nucleotide sequence of the coding version of SlcIl4resCD40LpORF is shown in FIG. 4A-4B.

### EXAMPLE 2

### Enhanced Immunogenicity of Human PSA using a Plasmid Adjuvant

In order to assess whether plasmid SlcIl4resCD40LpORF enhanced the immunogenicity of a coadministered antigen, in this case a nucleic acid construct encoding the human prostate-specific antigen (PSA), the following experiment was conducted. The PSA nucleic acid immunogen was produced as DNA fragments capable of expressing the PSA protein using a ligase-assisted PCR amplification method as described in commonly owned, copending patent application entitled “A High-Throughput Method of DNA Immunogen Preparation and Immunization” Attorney docket number 7037-0001), filed Nov. 26, 2004, incorporated herein by reference in its entirety.

Animal immunization was achieved electrically through the electroporation of leg tissues with the antigen-encoding DNA, essentially as described in Selby et al., J. Immunol. (2000) 164:4635-4640. Briefly, the TA (tibialis anterior) muscle regions of the two hind legs were shaved and 50 μl of the DNA fragments (5-15 microgram) in PBS (phosphate-buffered saline) were injected into each muscle. Using an electroporator from BTX Molecular Delivery Systems (ECM 830), electric shocks were delivered as: 100 volts; pulse length of 50 milliseconds, 200 millisecond pulse interval, 5 pulses total. Boost electroporation, performed identical to the primary immunization, was carried out 2 weeks after the primary. The mice were sacrificed 4 weeks later for antisera for analyses.

Five groups of Balb/c mice were also immunized via electroporation with 5 micrograms PSA (human prostate specific antigen)-expressing DNA fragments (prepared as described above) along with the adjuvant plasmid (FIG. 1C) or its non-coding variant (FIG. 1D) (15 microgram each) and boosted at 2 weeks. 4 weeks later the antisera were obtained and diluted 1:2000 and analyzed by ELISA for anti-PSA antibody titers using an ELISA PSA kit (Biocheck, Burlingame, Calif.). As explained above, the non-coding variant contained the cytokine segment in the opposite orientation and was therefore not expected to encode the cytokines.

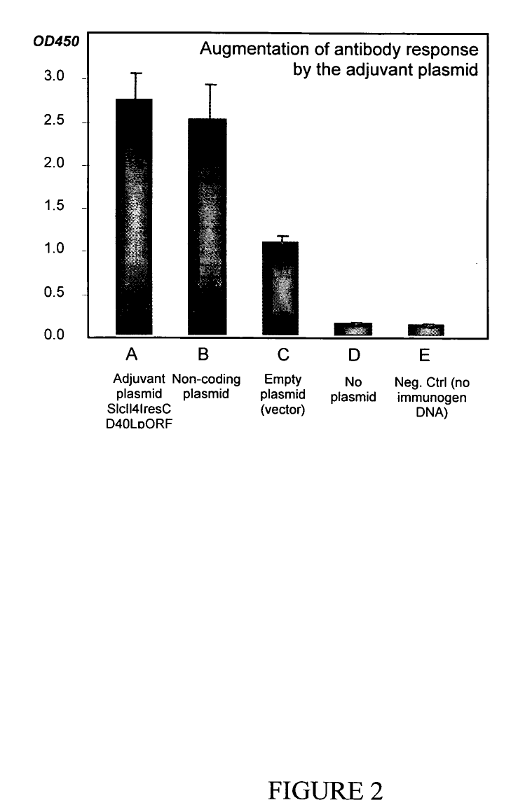

Results are shown in FIG. 2. A, B, C, D and E shown on the horizontal axis are as follows: (A) animals administered PSA constructs plus SlcIl4IresCD40LpORF (FIG. 1C); (B) animals administered PSA constructs and its non-coding variant that contains the cytokine segment in the opposite orientation and is therefore not expected to encode the cytokines (FIG. 1D); (C) animals given an empty plasmid that lacks the promoter and the cytokine segment; (D) animals given PSA without any plasmid DNA; and (E) animals that were immunized with a negative control containing no immunogen.

As can be seen, a comparison of groups A and D indicates that the adjuvant plasmid was highly effective in promoting antibody production (some 15 fold). Similarly, the non-coding counterpart was also effective in promoting antibody production. A comparison of groups A and B indicates a marginal but detectable effect of the cytokine proteins. A comparison of groups B and C suggests that sequences of SLC-IL4, IRES and/or CD40 ligand may be important in enhancing antibody response.

### EXAMPLE 3

### Enhanced Immunogenicity of Human MTF Using a Plasmid Adjuvant

The human MTF gene, normally encoding an intracellular protein, was used to examine the immune adjuvant effects of the plasmid SlcIl4IresCD40LpORF. The 3′ terminal 1.1 kb region of the MTF open reading frame (MTFC) was amplified by PCR and fused to a secretory signal sequence encoding the N-terminal 20 amino acids from the murine Ig-kappa gene. Expression DNA fragments were produced using a ligation-assisted DNA amplification method as described in commonly owned, copending provisional patent application entitled “A High-Throughput Method of DNA Immunogen Preparation and Immunization” Attorney docket number 7037-0001p), filed Nov. 26, 2003, incorporated herein by reference in its entirety. The DNA fragments were expected to express MTFC at the extracellular space in order to maximize the interaction of the MTFC protein with the immune system.

Immunization was carried out using these fragments and the plasmid adjuvants using procedures similar to those described above. In particular, 10 μg MTFC-expressing DNA fragments and 10 μg of the adjuvant plasmids were co-electroporated into each Balb/c mouse at the primary or the boost immunization. The antisera were analyzed using an ELISA as described above except that the antigen in the assay was purified MTFC fused with GST (glutathione-S-transferase) bound to glutathione-coated beads.

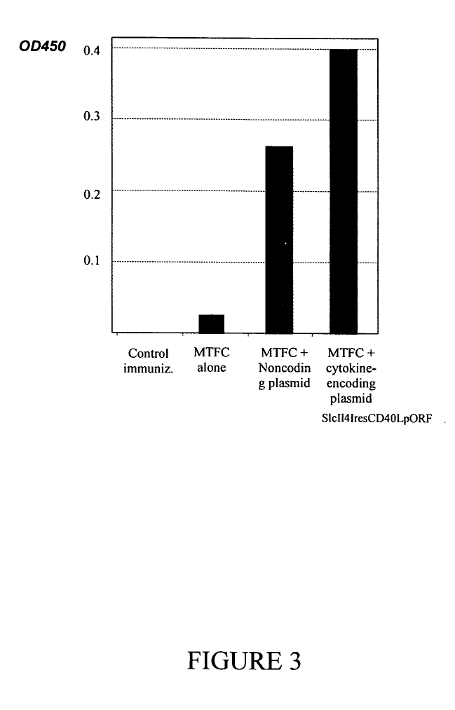

As shown in FIG. 3 and consistent with the findings in the PSA experiment, both the cytokine-encoding plasmid SlcIl4IresCD40LpORF and its non-coding version exhibited marked immunity-boosting function. The cytokine-encoding plasmid showed a larger adjuvant effect than the non-coding plasmid.

Thus, plasmid adjuvants, as well as methods for producing and using the same to enhance immunogenic responses and antibody production are described. Although preferred embodiments of the subject invention have been described in some detail, it is understood that obvious variations can be made without departing from the spirit and the scope of the invention as defined herein.

1. An adjuvant plasmid, comprising in 5′ to 3′ order:
a) a promoter sequence,
b) a sequence encoding a chemokine/cytokine fusion protein,
c) an internal ribosome entry sequence (IRES), and
d) a sequence encoding a CD40 ligand.

2. The adjuvant plasmid of claim 1, comprising the polynucleotide sequence of SEQ ID NO:1.

3. The adjuvant plasmid of claim 1, comprising a polynucleotide sequence having at least 75% sequence identity to the contiguous sequence of SEQ ID NO:1.

4. The adjuvant plasmid of claim 1, wherein the CD40 ligand is secreted or membrane-bound.

5. The adjuvant plasmid of claim 1, wherein the sequence encoding the chemokine/cytokine fusion protein comprises the sequence of SEQ ID NO:2.

6. The adjuvant plasmid of claim 1, wherein the sequence encoding the chemokine/cytokine fusion protein comprises a sequence encoding a chemokine fused to the 3′ terminus of a sequence encoding a cytokine.

7. The adjuvant plasmid of claim 1, wherein the sequence encoding the chemokine/cytokine fusion protein comprises a sequence encoding a chemokine fused to the 5′ terminus of a sequence encoding a cytokine.

8. The adjuvant plasmid of claim 1, wherein the sequence encoding the chemokine/cytokine fusion protein comprises a sequence encoding a cytokine selected from the group consisting of IL-2, IL-3, IL-4, IL-5, IL-6, IL-9, IL-10, IL-13, IL-21, IL-25, GM-CSF, IFN-γ, and TNF-α.

9. The adjuvant plasmid of claim 1, wherein the sequence encoding the chemokine/cytokine fusion protein comprises a sequence encoding SLC fused to the 5′ terminus of a sequence encoding a cytokine.

10. The adjuvant plasmid of claim 9, wherein the sequence encoding the chemokine/cytokine fusion protein comprises a sequence encoding SLC fused to the 5′ terminus of a sequence encoding IL-4.

11. The adjuvant plasmid of claim 1, wherein the sequence encoding the chemokine/cytokine fusion protein comprises a sequence encoding SLC fused to the 3′ terminus of a sequence encoding a cytokine.

12. The adjuvant plasmid of claim 11, wherein the sequence encoding the chemokine/cytokine fusion protein comprises a sequence encoding SLC fused to the 3′ terminus of a sequence encoding IL-4.

13. The adjuvant plasmid of claim 1, wherein the promoter sequence is operably linked to the sequence encoding the chemokine/cytokine fusion protein.

14. The adjuvant plasmid of claim 13, wherein the promoter sequence is selected from the group consisting of:
a) an SV40 promoter,
b) a CMV promoter,
c) a mouse mammary tumor virus LTR promoter,
d) an adenovirus major late promoter,
e) a herpes simplex virus promoter,
f) an EF1 alpha promoter, and
g) a promoter derived from the murine metallothionein gene.

15. The adjuvant plasmid of claim 14, further comprising at least one control element selected from the group consisting of a transcription enhancer element, a transcription termination signal, a UTR sequence, a polyadenylation sequence, a sequence for optimization of initiation of translation, and a translation termination sequence.

16. The adjuvant plasmid of claim 15, wherein the transcription enhancer element is selected from the group consisting of:
a) an SV40 enhancer element,
b) a LTR derived enhancer element,
c) a Rous Sarcoma Virus enhancer element, and
d) a CMV enhancer element.

17. The adjuvant plasmid of claim 15, wherein the transcription termination signal is selected from the group consisting of:
a) an SV40 transcription termination signal, and
b) a bovine growth hormone transcription termination signal.

18. A non-coding adjuvant plasmid, comprising in 5′ to 3′ order:
a) a promoter sequence,
b) a sequence comprising a sequence encoding a chemokine/cytokine fusion protein reversed in anti-sense orientation,
c) an internal ribosome entry sequence (IRES) reversed in anti-sense orientation, and
d) a sequence encoding a CD40 ligand.

19. The non-coding adjuvant plasmid of claim 18, wherein the plasmid comprises the polynucleotide sequence of SEQ ID NO:4.

20. The non-coding adjuvant plasmid of claim 18, wherein the plasmid comprises a polynucleotide sequence having at least 75% sequence identity to the contiguous sequence of SEQ ID NO:4.

21. A plasmid adjuvant system comprising:
a) a polynucleotide comprising a sequence encoding SLC,
b) a polynucleotide comprising a sequence encoding a cytokine, and
c) a polynucleotide comprising a sequence encoding a CD40-ligand.

22. The plasmid adjuvant system of claim 21, wherein the cytokine is selected from the group consisting of IL-2, IL-3, IL-4, IL-5, IL-6, IL-9, IL-10, IL-13, IL-21, IL-25, GM-CSF, IFN-γ, and TNF-α.

23. The plasmid adjuvant system of claim 21, wherein the plasmid adjuvant system comprises:
a) a plasmid comprising the polynucleotide comprising a sequence encoding SLC,
b) a plasmid comprising the polynucleotide comprising a sequence encoding a cytokine, and
c) a plasmid comprising the polynucleotide comprising a sequence encoding a CD40-ligand.

24. The plasmid adjuvant system of claim 21, wherein the plasmid adjuvant system comprises:
a) a plasmid comprising:
(i) the polynucleotide comprising a sequence encoding SLC, and
(ii) the polynucleotide comprising a sequence encoding a cytokine, and
b) a plasmid comprising the polynucleotide comprising a sequence encoding a CD40-ligand.

25. The plasmid adjuvant system of claim 21, wherein the plasmid adjuvant system comprises:
a plasmid comprising:
a) a polynucleotide comprising a sequence encoding SLC,
b) a polynucleotide comprising a sequence encoding a cytokine, and
c) a polynucleotide comprising a sequence encoding a CD40-ligand.

26. The plasmid adjuvant system of claim 21, further comprising a polynucleotide encoding an immunogen.

27. The plasmid adjuvant system of claim 26, wherein the polynucleotide encoding an immunogen is derived from an organism selected from the group consisting of:
a) a bacteria,
b) a mycobacteria,
c) a virus,
d) a fungus, and
e) a parasite.

28. A composition comprising the adjuvant plasmid of claim 1.

29. A composition comprising the non-coding adjuvant plasmid of claim 18.

30. A composition comprising the adjuvant plasmid system of claim 21.

31. The composition of any of claims 28-30, further comprising an immunogen.

32. The composition of claim 31, wherein the immunogen is selected from the group consisting of polynucleotides, polypeptides, inactivated or attenuated pathogens, glycoproteins, polysaccharides, and lipids.

33. The composition of claim 31, further comprising an adjuvant.

34. The composition of claim 31, further comprising a pharmaceutically acceptable excipient.

35. The composition of claim 31, further comprising an immunomodulatory molecule.

36. The composition of claim 35, wherein the immunomodulatory molecule is selected from the group consisting of B7-1, B7-2, GM-CSF, IL-2, and IL-12.

37. A method of eliciting an immunological response in a vertebrate subject, comprising administering the composition of claim 31 to said subject.

38. A method of eliciting an immunological response in a vertebrate subject, comprising:
administering a composition comprising an immunogen to a vertebrate subject, and coadministering the composition of any of claims 28-30 to said vertebrate subject.

39. A method of making a polyclonal antibody, the method comprising:
a) coadministering an immunogen and an adjuvant plasmid of claim 1 or a non-coding adjuvant plasmid of claim 18 or an adjuvant plasmid system of claim 21 to an animal under conditions that permit the expression of said adjuvant plasmid or plasmid system, thereby eliciting an antibody response to said immunogen in said animal,
b) isolating antibodies from the animal, and
c) screening the isolated antibodies with said immunogen, thereby identifying a polyclonal antibody which specifically binds to said immunogen.

40. A method of making a monoclonal antibody, the method comprising:
a) coadministering an immunogen and an adjuvant plasmid of claim 1 or a non-coding adjuvant plasmid of claim 18 or an adjuvant plasmid system of claim 21 to an animal under conditions that permit the expression of said adjuvant plasmid or plasmid system, thereby eliciting an antibody response to said immunogen in said animal,
b) isolating antibody producing cells from the animal,
c) fusing the antibody producing cells with immortalized cells to form monoclonal antibody-producing hybridoma cells,
d) culturing the hybridoma cells, and
e) isolating from the culture a monoclonal antibody which specifically binds to said immunogen.


SEQUENCE LISTING (4 sequences)

SEQ ID NO: 1 - DNA, 5365 bp, Artificial
  Cytokine encoding version of plasmid SlcIl4IresCD40LpORF
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagaggaggccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtagatctcgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggccatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 2 - DNA, 769 bp, Artificial
  nucleotide sequence of a representative SLC-IL-4 fusion
atggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagaggaggccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtagatct

SEQ ID NO: 3 - protein, 254 aa, Artificial
  amino acid sequence of a representative SLC-IL-4 fusion
MAQMMTLSLLSLVLALCIPWTQGSDGGGQDCCLKYSQKKIPYSIVRGYRKQEPSLGCPIPAILFSPRKHSKPELCANPEEGWVQNLMRRLDQPPAPGKQSPGCRKNRGTSKSGKKGKGSKGCKRTEQTQPSRGGHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 4 - DNA, 5365 bp, Artificial
  Non-coding adjuvant plasmid
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggtattatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgagatctacgagtaatccatttgcatgatgctctttaggctttccaggaagtctttcagtgatgtggacttggactcattcatggtgcagcttatcgatgaatccaggcatcgaaaagcccgaaagagtctctgcagctccatgagaacactagagttcttcttcaagcatggagttttcccatgttttaaataaaatatgcgaagcaccttggaagccctacagacgagctcactctctgtggtgttcttcgttgctgtgaggacgtttggcacatccatctccgtgcatggcgtcccttctcctgtgacctcgttcaaaatgccgatgatctctctcaagtgatttttgtcgcatccgtggatatggcctcctcttgagggctgtgtctgttcagttctcttgcagcccttggagccctttcctttctttccagacttagaggttccccggttcttcctgcagccggggctttgtttccctggggctggaggctggtccaggcggcgcatcaggttctgcacccagccttcctcagggtttgcacatagctcaggcttagagtgcttccggggtgagaacaggattgccgggatgggacagcctaaacttggttcttgcttcctatagcctcggacaatactgtagggaattttcttctggctgtacttaaggcagcagtcctgaccccctccatcactgccttgggtccaggggatgcagagagccaggaccaggctaaggaggctcagagtcatcatctgagccatggccatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa